(19) United States
(12) Patent Application Publication
(10) Pub. No.: US 2011/0251456 A1
(43) Pub. Date: Oct. 13, 2011

(54) Method and Apparatus For Viewing A Body Cavity

(76) Inventors: Stephen C. Jacobsen, Salt Lake City, UT (US); Fraser M. Smith, Salt Lake City, UT (US); David P. Marceau, Salt Lake City, UT (US); David T. Markus, Salt Lake City, UT (US)

(21) Appl. No.: 12/896,737

(22) Filed: Oct. 1, 2010

Related U.S. Application Data
(60) Provisional application No. 61/247,883, filed on Oct. 1, 2009.

(51) Int. Cl. A61B 1/04

(52) U.S. Cl. 600/109

(57) ABSTRACT

A method and apparatus to generate a planar representation of a longitudinally extending 360 degree continuous view within a body cavity of a patient is disclosed comprising advancing a portion of an imaging device into the body cavity of the patient, the imaging device having an image capture mechanism disposed on a distal end thereof configured to capture at least a 360 degree view of the inside of the body cavity. Further comprising withdrawing the imaging device at a controlled rate from the patient while simultaneously coordinating and generating 360 degree view image data from the imaging device and transmitting the image data from the imaging device to an image processor. The method further comprising processing the image data to produce a planar longitudinally continuous 360 degree view of the body cavity.

### CLAIM OF PRIORITY

This application claims priority to U.S. Provisional Application No. 61/247,883 filed on Oct. 1, 2009 which is incorporated herein by reference in its entirety.

### FIELD OF THE INVENTION

The present invention relates to medical devices, and more particularly to miniaturized in-situ imaging devices and methods of operation of said devices.

### BACKGROUND

Minimally invasive diagnostic medical procedures are used to assess the interior surfaces of an organ by inserting a tube into the body. The instruments utilized may have a rigid or flexible tube and provide an image for visual inspection and photography, but also enable taking biopsies and retrieval of foreign objects. Analysis of image data collected during the inspection and photography of the interior of the body cavity is a critical component of proper diagnosis of disease and other related conditions.

### SUMMARY OF THE INVENTION

One exemplary embodiment of the invention provides a medical imaging device comprising an elongated cylindrical member configured for insertion into a patient. The elongated cylindrical member has a distal end and a proximal end, a plurality of SSIDs disposed at the distal end of the elongated cylindrical member, a plurality of lenses in contact with the plurality of SSIDs, and an annular prism optically coupled to the plurality of lenses.

In another exemplary embodiment of the invention, a medical device is provided comprising an elongated cylindrical member configured for insertion into a patient having a proximal end and a distal end. The device further comprises at least one SSID disposed at the distal end of the elongated cylindrical member, wherein the image plane of the SSID is oriented substantially parallel to a longitudinal axis of the elongated cylindrical member. The device further has at least one lens disposed on the SSID and a rotation mechanism coupled to the at least one SSID for rotating the SSID about an axis substantially parallel to a longitudinal axis of the elongated cylindrical member.

In another exemplary embodiment of the invention, a method of generating a planar image of a longitudinally extending 360 degree continuous view within a body cavity of a patient is disclosed comprising advancing a portion of an imaging device into the body cavity of the patient, the imaging device having an image capture mechanism disposed on a distal end thereof configured to capture at least a 360 degree view of the inside of the body cavity. The method further comprises withdrawing the portion of the imaging device at a controlled rate from the patient while simultaneously coordinating and generating 360 degree view image data from the imaging device and transmitting the image data from the imaging device to an image processor. The method further comprises processing the image data to produce a planar longitudinally continuous 360 degree view of the body cavity.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 is a medical device in accordance with one embodiment of the present invention;

FIG. 2 is a cross-sectional view of the distal end of the medical device of FIG. 1;

FIG. 3 is a perspective view of an annular prism in accordance with one embodiment of the present invention;

FIG. 4 is a perspective view of a substrate having a plurality of SSIDs according to one embodiment;

FIG. 5 is a perspective view of the substrate of FIG. 4 having a lens system optically coupled to the SSIDs in accordance with one embodiment of the present invention;

FIG. 6 is a top view of the annular prism of FIG. 3;

FIG. 7 is a top view of the substrate of FIG. 4;

FIG. 8 is a top view of the substrate of FIG. 6;

FIG. 9 is a cross-sectional view of one embodiment of a medical imaging device according to one embodiment of the present invention;

FIG. 10 is a cross-sectional view of one embodiment of a medical imaging device;

FIG. 11 is a front view of one embodiment of a medical imaging device showing one example of an image capture area;

FIG. 12 is a cross-section of a medical imaging device showing one example of an image capture area;

FIG. 13 is side view of a medical imaging device showing one example of an image capture area;

FIG. 14 is a side view of a medical imaging device showing one example of an image capture area;

FIG. 15 is an exemplary 360 degree view image in accordance with one embodiment of the present invention;

FIG. 16 is an exemplary longitudinally continuous 360 degree view in accordance with one embodiment of the invention;

FIG. 17 is an exemplary planar representation of the longitudinally continuous 360 degree view of FIG. 16;

FIG. 18 is a depiction of a planar representation of a longitudinally continuous 360 degree view of an image in accordance with one embodiment of the present invention;

FIG. 19 is a perspective view of a single SSID with a single imaging array disposed thereon in accordance with one embodiment of the present invention; and

FIG. 20 is a perspective view of the single SSID of FIG. 19 with an annular prism and lens disposed within the center of the annular prism in accordance with one embodiment of the present invention.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

It must be noted that, as used in this specification and the appended claims, singular forms of “a,” “an,” and “the” include plural referents unless the context clearly dictates otherwise.

An “SSID,” “solid state imaging device,” “SSID chip,” or “solid state imaging chip” in the exemplary embodiments generally comprises an imaging array or pixel array for gathering image data. In one embodiment, the SSID can comprise a silicon or other semiconductor substrate or amorphous silicon thin film transistors (TFT) having features typically manufactured therein. Features can include the imaging array, conductive pads, metal traces, circuitry, etc. Other integrated circuit components can also be present for desired applications. However, it is not required that all of these components be present, as long as there is a means of gathering visual or photon data, and a means of sending that data to provide a visual image or image reconstruction.

The term “umbilical” can include the collection of utilities that operate the SSID or the micro-camera as a whole. Typically, an umbilical includes a conductive line, such as electrical wire(s) or other conductors, for providing power, ground, clock signal, and output signal with respect to the SSID, though not all of these are strictly required. For example, ground can be provided by another means than through an electrical wire, e.g., to a camera housing such as micromachined tubing, etc. The umbilical can also include other utilities such as a light source, temperature sensors, force sensors, fluid irrigation or aspiration members, pressure sensors, fiber optics, microforceps, material retrieval tools, drug delivery devices, radiation emitting devices, laser diodes, electric cauterizers, and electric stimulators, for example. Other utilities will also be apparent to those skilled in the art and are thus comprehended by this disclosure.

“GRIN lens” or “graduated refractive index lens” refers to a specialized lens that has a refractive index that is varied radially from a center optical axis to the outer diameter of the lens. In one embodiment, such a lens can be configured in a cylindrical shape, with the optical axis extending from a first flat end to a second flat end. Thus, because of the differing refractive index in a radial direction from the optical axis, a lens of this shape can simulate the effects of a more traditionally shaped lens. The GRIN lens may be a GRIN rod lens or any other GRIN lens configuration.

With these definitions in mind, reference will now be made to the accompanying drawings, which illustrate, by way of example, embodiments of the invention.

Use of imaging devices within portions of a patient can be particularly useful in medical diagnostic and treatment applications. For example, portions of human anatomy previously viewable only by a surgical procedure can be viewed now by minimally invasive procedures, provided an imaging device can be made that is small enough to view the target anatomy. Further, many medical imaging tools designed to be placed within the body of a patient require significant residence time within the patient to properly diagnose an ailment. Other tools provide only a static or limited view of the internal cavity of the patient.

Advantageously, in one embodiment of the present invention, creating a three-dimensional continuous digital image of a body cavity invention allows the medical practitioner to quickly image a body cavity of a patient and thereafter analyze the image from multiple points of view for further diagnosis of the patient. A prompt scan of the body cavity of the patient minimizes the amount of time a patient must endure the procedure. While the present invention has applications in these aforementioned fields and others, the medical imaging application can be used to favorably illustrate unique advantages of the invention.

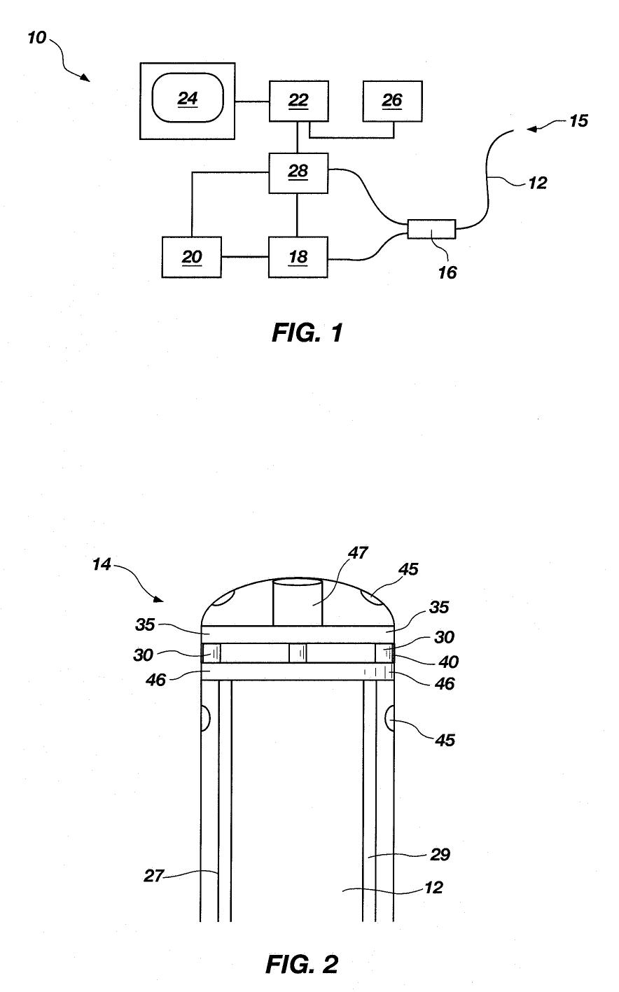

With reference to FIGS. 1 and 2, in one embodiment of the present invention, a medical imaging system 10 comprises a micro-catheter 12 having an imaging device, shown generally at 14, disposed at a distal tip 15 of the micro-catheter 12. A processor 22, such as an appropriately programmed computer, is provided to control the imaging system 10 and create an image of anatomy adjacent the distal tip portion 15, within a patient (not shown), displayable on a monitor 24, and storable in a data storage device 26. An interface 28 is provided which supplies power to the imaging device 14 and feeds a digital image signal to the processor based on a signal received from the imaging device via an electrical umbilical 27, including conductive wires 29 through the micro-catheter 12. A light source may also be provided at the distal end of the micro-catheter 12. In one aspect, the system further includes a fitting 16 enabling an imaging fluid, such as a clear saline solution, to be dispensed to the distal tip portion of the micro-guidewire from a reservoir 18 through an elongated tubular member (not shown) removably attached to the micro-guidewire to displace body fluids as needed to provide a clearer image. A pump 20 is provided, and is manually actuated by a medical practitioner performing a medical imaging procedure, or can be automated and electronically controlled so as to dispense fluid on demand according to control signals from the practitioner, sensors, or according to software commands. Additional principles of operation and details of construction of similar imaging device assemblies can be found in U.S. patent application Ser. Nos. 10/391,489, 10/391,490, 11/292,902, 10/391,513, and 11/810,702 each of which are incorporated herein by reference in their entireties.

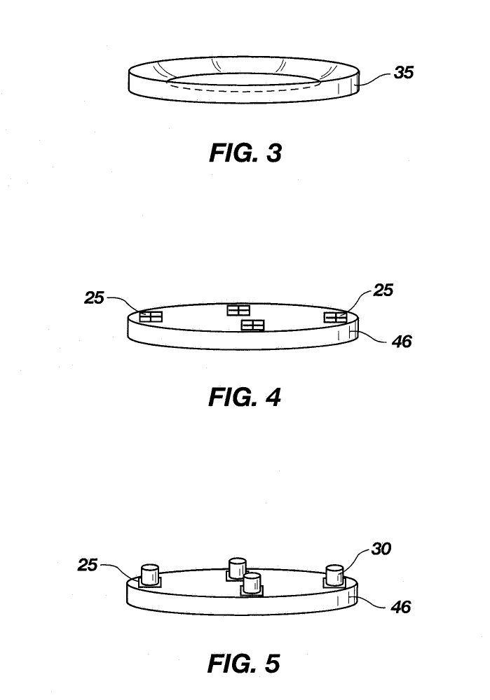

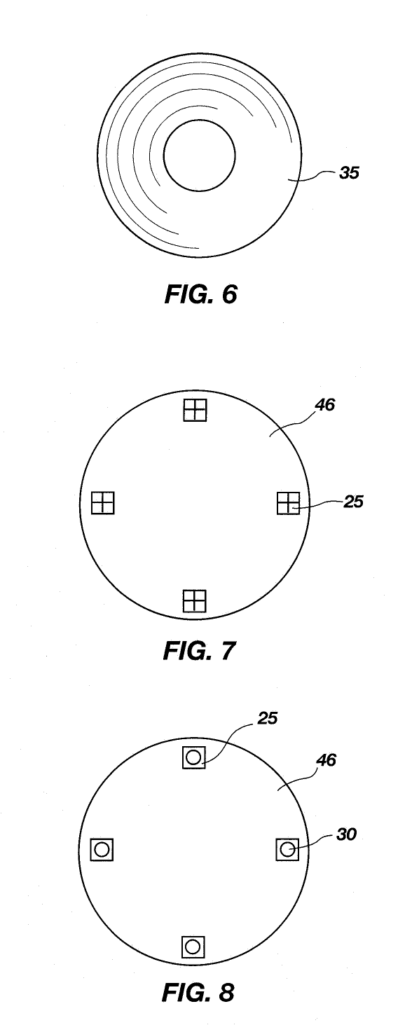

Referring now to FIGS. 2-8, a micro-catheter 12 is provided having a plurality of SSIDs 25 disposed at the distal tip 15 of the micro-catheter 12. A plurality of lenses 30 are in contact with the plurality of SSIDs 25 and an annular prism 35 is optically coupled to the plurality of lenses 30. An annular optical window 40 is provided about a perimeter of the micro-catheter 12 corresponding to the annular prism 35. Light from within the body cavity is collected through the optical window 40 and directed to the plurality of lenses 30 and SSIDs 25 via the annular prism 35. In one aspect of the invention, light is emitted from the distal tip 15 of the micro-catheter 12 through at least one light emitting member 45.

In another embodiment of the present invention, the plurality of SSIDs 25 are disposed on a cylindrical substrate 46 having a diameter approximately identical to the inner diameter of the micro-catheter 12. Example SSIDs contemplated for use in one embodiment of the present invention include charge coupled devices (CCDs), three-CCD devices having three separate CCDs, each one taking a separate measurement of red, green, and blue light (3CCDs), and/or complementary metal-oxide-semiconductors (CMOSs). In one embodiment, the SSIDs 25 are oriented about a perimeter of the substrate 46 with their image plane oriented substantially parallel to the substrate 46. However, it is understood that the SSIDs 25 can be placed anywhere on the substrate 46 with the image plane oriented in any appropriate direction to suit the particular application. For example, an additional SSID may be placed at the center of the substrate 46 with an appropriate lens system 47 disposed thereon for collecting image data in the direction of the distal tip 15 of the micro-catheter 12.

In one embodiment of the invention, the lens system 30 can comprise a plurality of GRIN lenses oriented to transmit an image on the corresponding image planes of the SSIDs 25. However, it is understood that any appropriate lens system capable of directing the image from the annular prism 35 to the SSIDs 25 is contemplated herein.

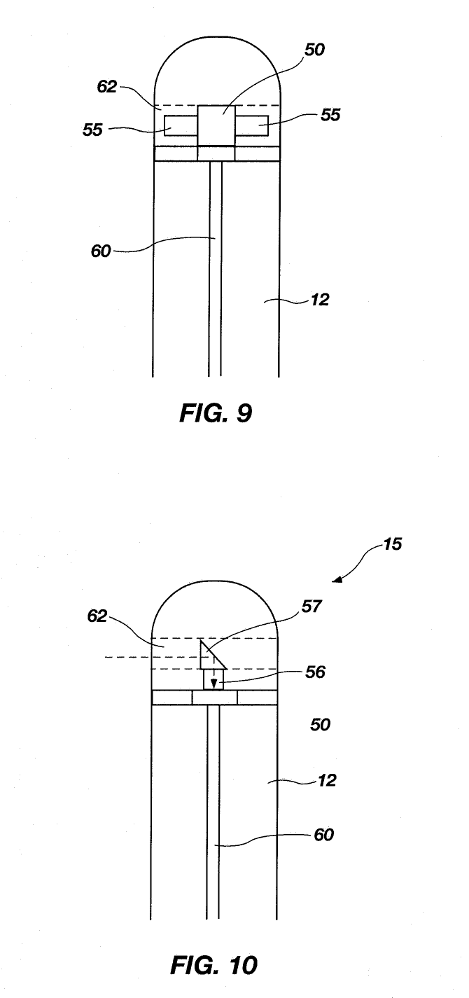

Referring now to FIG. 9, in another embodiment of the present invention, a micro-catheter 12 is provided having at least one SSID 50 disposed at the distal end of the micro-catheter 12. The image plane of the SSID 50 is oriented substantially non-parallel to a longitudinal axis of the micro-catheter 12. At least one lens 55 is disposed on the SSID 50. In one aspect, the lens is a GRIN lens optically coupled to the SSID 50. The micro-catheter 12 further has a rotation mechanism 60 coupled to the at least one SSID 50 for rotating the SSID 50 about an axis substantially parallel to a longitudinal axis of the micro-catheter 12. In another embodiment, the micro-catheter 12 comprises a plurality of SSIDs 50 wherein the image plane of each of the SSIDs 50 is oriented substantially parallel to a longitudinal axis of the micro-catheter 12.

Referring to FIG. 10, in another embodiment, a micro-catheter 12 has at least one SSID 50 disposed at the distal end of micro-catheter 12 having a GRIN lens 56 disposed thereon and a prism 57 disposed on a distal end of the GRIN lens 56. The micro-catheter 12 has a rotation mechanism 60 coupled to the at least one SSID 50 for rotating the SSID 50 about an axis substantially parallel to a longitudinal axis of the micro-catheter 12. As the rotation mechanism 60 rotates the SSID 50 about the axis, light is received through an annular optical window 62 and transmitted through the prism 57, the GRIN lens 56, and to the SSID 50. In this manner, a 360-degree image of a portion of a body cavity may be collected. Conductive lines (not shown) provide power to the imaging device and also provide a means for transmitting the image data to a data processor and display.

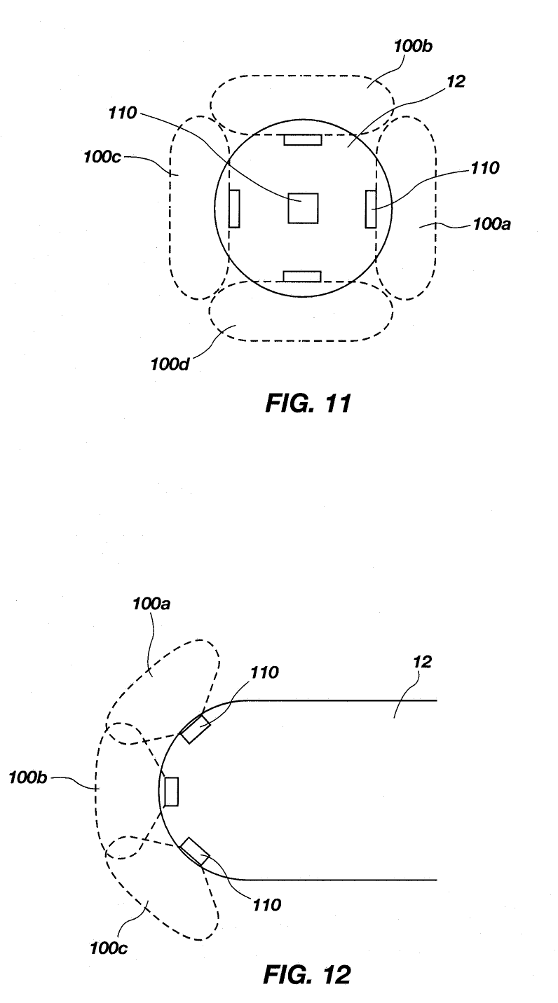

Referring generally to FIGS. 11 and 12, in accordance with another embodiment of the invention, a method of generating a planar image of a longitudinally extending 360 degree continuous view within a body cavity of a patient is disclosed comprising advancing a micro-catheter 12 into the body cavity of the patient wherein the micro-catheter 12 has an image capture mechanism 110 disposed on a distal end thereof. The image capture mechanism 110 is configured to capture at least a 360 degree view of the inside of the body cavity. The method further comprises withdrawing the micro-catheter 12 from the patient at a controlled rate while simultaneously coordinating and generating 360 degree view image data from the imaging capture mechanism 110. In one aspect of the invention, the image capture mechanism 110 comprises a plurality of SSIDs with a lens system as shown in FIGS. 2-8 as described herein. While specific reference is made to the imaging device disclosed herein, it is understood that any device capable of capturing a 360 degree view of a body cavity is contemplated for use herein.

Following collection of the image data, the image data is transmitted from the imaging capture mechanism 110 to an image processor 22, as illustrated in FIG. 1, wherein the image data is processed to produce a planar longitudinally continuous 360 degree view of the body cavity. In essence, the entire inside of the body cavity subject to the imaging may be displayed as a planar image. In one exemplary embodiment, a planar representation of the longitudinally continuous 360 degree view of the body cavity is accomplished by tiling or seamlessly integrating the images captured from the individual capture area of one or more of the imaging devices 110. As illustrated in FIGS. 11 and 12, in one embodiment, the planar representation comprises a composite of the images from, for example, image capture areas 100a, 100b, 100c, and 100d.

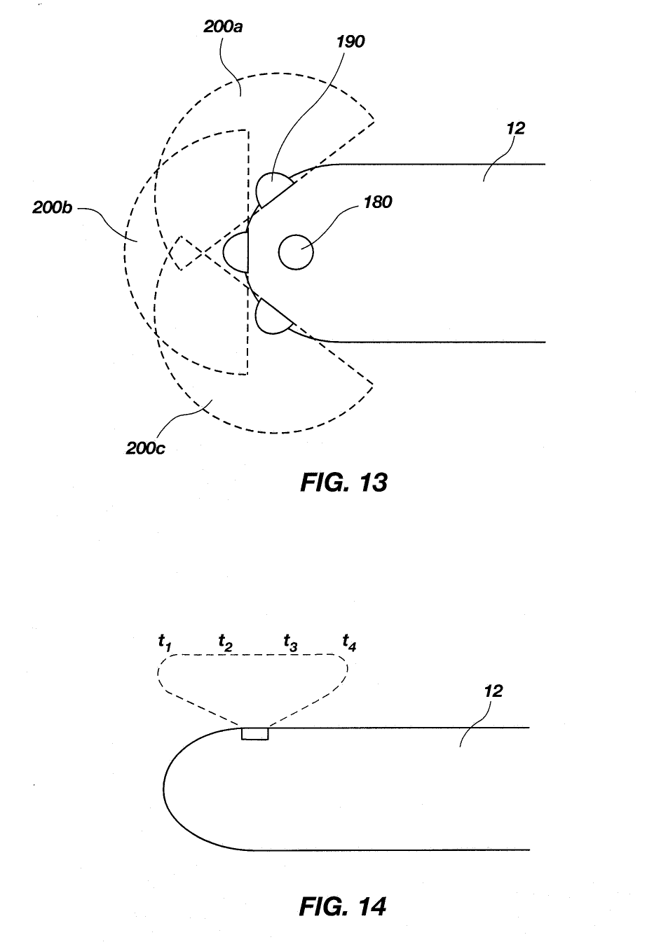

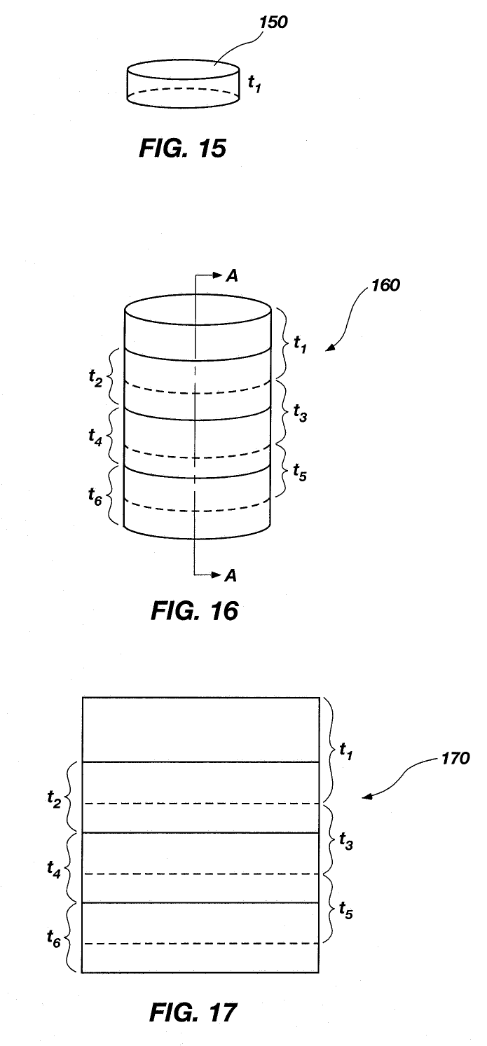

Referring now to FIGS. 14-17, as the micro-catheter 12 is withdrawn or advanced within the body cavity, the 360 degree view created by tiling images from the imaging devices 110 from image capture areas 100a, 100b, 100c, and 100d are further tiled together over time to create a longitudinally continuous 360 degree view of the body cavity. By way of example, and without limitation, an initial 360 degree view may be thought of as creating an annular image 150 at time=1 as illustrated in FIG. 15. As noted above, annular image 150 is a composite of images from image capture areas 100a, 100b, 100c, and 100d. As the micro-catheter 12 is withdrawn or advanced within the body cavity, the annular image 150 at time=1 is extended in the direction of travel of the micro-catheter to create a cylindrical image 160. The cylindrical image 160 is a composite of a plurality of annular images 150 taken at time=1 through time=6, respectively. The cylindrical image 160 can be processed further, using appropriate image correction techniques, to transform the cylindrical image into a planar representation 170 of the interior of the body cavity scanned. In one exemplary embodiment, FIG. 17 shows a planar representation 170 of the cylindrical image 160 of FIG. 16 wherein the cylindrical image 160 has been “opened up” along line A-A′. While specific reference is made herein regarding the order in which the image capture areas are tiled together, such reference is exemplary, as the images may be tiled together in any order to achieve the desired planar representation. Advantageously, a medical practitioner, or other user, may scan the interior of a body cavity and thereafter view the entire interior of the body cavity on a flat display. Referring to FIG. 13, in yet another embodiment, the 360 degree view of the body cavity may be captured with the use of one or more fisheye lenses 190. The image capture area 200a, 200b, 200c of each of the fisheye lenses 190 can also be tiled together to create a 360 degree view and can also be used to create the longitudinally continuous 360 degree view.

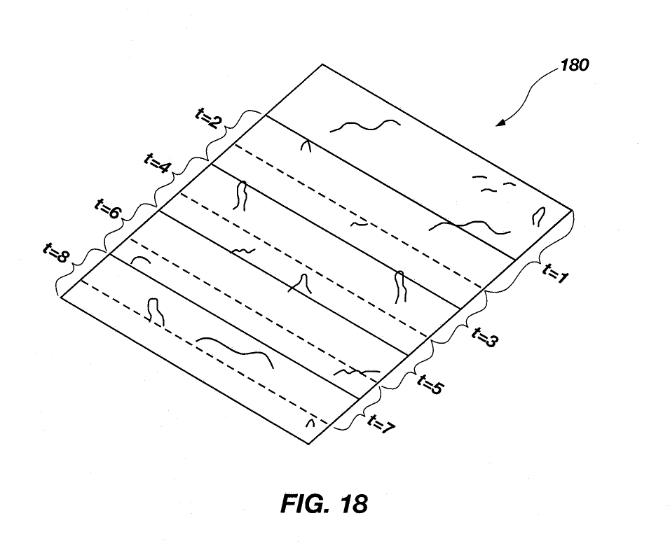

Referring now to FIG. 18, in another embodiment of the present invention, the method further comprises processing the image data to produce a three-dimensional representation of the inside of the body cavity. Advantageously, the three-dimensional representation allows a medical practitioner, or other user, to digitally navigate the three-dimensional representation thereby viewing portions of the inside of the body cavity from different points of view. This allows the user to further examine and diagnose illness, malady, or other conditions, within the body cavity. Similar to the method noted above, the three-dimensional image may also be “opened up” to show a quasi-planar three-dimensional representation 180 of the interior of the body cavity which is scanned. As with the planar representation described in FIGS. 15-17, FIG. 18 comprises a depiction of an example composite three-dimensional image of the interior of a body cavity. While use of the aforementioned medical devices is contemplated herein as the imaging device capable of capturing at least a 360 degree view of the inside of the body cavity, use of magnetic resonance imaging devices, ultrasound imaging devices, interferometry devices, or other suitable imaging devices, or a combination of suitable imaging devices is contemplated herein.

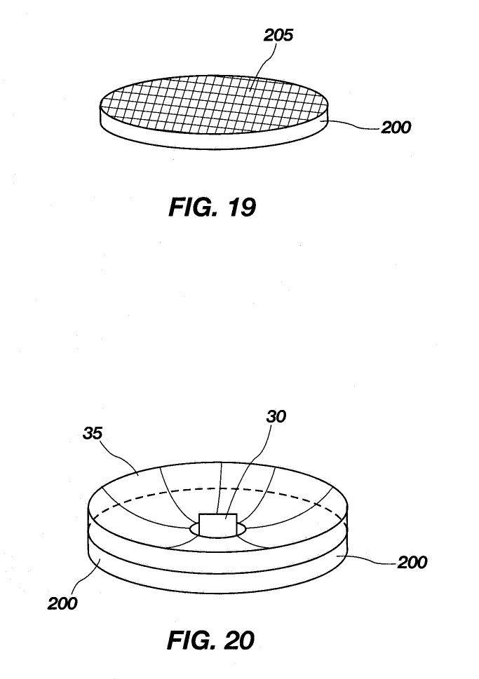

With reference now to FIGS. 1, 19, and 20, in accordance with another embodiment of the present invention, a micro-catheter 12 may be equipped with a single SSID 200 on a distal tip 15 of the micro-catheter. An annular prism 35 may be disposed directly on a top surface of the SSID 200, wherein the SSID 200 comprises a single imaging array 205. A single lens 30 may be placed in the center of the annular prism 35 to assist in imaging in a forward direction. In one aspect of the invention, a top surface of the annular prism 35 is coated with an opaque material to preclude interference with the imaging process and the single lens 30 further comprises a fish-eye lens.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A medical imaging device, comprising:
an elongated cylindrical member configured for insertion into a patient, the elongated cylindrical member having a distal end and a proximal end;
a plurality of solid state imaging chips disposed at the distal end of the elongated cylindrical member;
a plurality of lenses in contact with the plurality of SSIDs; and
an annular prism optically coupled to the plurality of lenses.

2. The medical imaging device of claim 1, further comprising an annular optical window corresponding to the annular prism.

3. The medical imaging device of claim 1, wherein the distal end of the device has at least one lumen therein.

4. The medical imaging device of claim 3, wherein the plurality of solid state imaging chips are disposed on a cylindrical substrate having a diameter approximately identical to the inner diameter of the at least one lumen.

5. The medical imaging device of claim 1, further comprising a light source emanating from a distal portion of the elongated cylindrical member.

6. The medical imaging device of claim 1, wherein the solid state imaging chip is selected from the group consisting of a CCD, a CMOS, and a 3CCD.

7. The medical imaging device of claim 1, wherein the elongated cylindrical member is operatively coupled to a data processor and storage device.

8. The medical imaging device of claim 1, wherein the image plane of the lenses is approximately perpendicular to the longitudinal axis of the elongated cylindrical member.

9. The medical imaging device of claim 1, wherein the plurality of lenses are GRIN lenses.

10. The medical imaging device of claim 1, wherein the plurality of lenses are fisheye lenses.

11. A medical imaging device, comprising:
an elongated cylindrical member configured for insertion into a patient having a proximal end and a distal end;
at least one solid state imaging chip disposed at the distal end of the elongated cylindrical member, wherein the image plane of the solid state imaging chip is oriented substantially parallel to a longitudinal axis of the elongated cylindrical member;
at least one lens disposed on the solid state imaging chip; and
a rotation mechanism coupled to the at least one solid state imaging chip for rotating the solid state imaging chip about an axis substantially parallel to a longitudinal axis of the elongated cylindrical member.

12. The medical imaging device of claim 11, wherein the at least one lens is a GRIN lens.

13. The medical imaging device of claim 11, further comprising a plurality of solid state imaging chips wherein the image plane of each of the solid state imaging chips is oriented substantially parallel to a longitudinal axis of the elongated cylindrical member.

14. A method of generating a planar image of a longitudinally extending 360 degree continuous view within a body cavity of a patient, comprising:
advancing a portion of an imaging device into the body cavity of the patient, the imaging device having an image capture mechanism disposed on a distal end thereof configured to capture at least a 360 degree view of the inside of the body cavity;
withdrawing the portion of the imaging device at a controlled rate from the patient while simultaneously coordinating and generating 360 degree view image data from the imaging device;
transmitting the image data from the imaging device to an image processor; and
processing the image data to produce a planar longitudinally continuous 360 degree view of the body cavity.

15. The method of claim 14, further comprising further processing the image data to produce a three-dimensional representation of the inside of the body cavity.

16. The method of claim 15, further comprising digitally navigating the three-dimensional representation thereby viewing portions of the inside of the body cavity from different points of view.

17. The method of claim 15, further comprising displaying the processed image data on an image display device.

18. The method of claim 14, wherein the image capture mechanism comprises at least one solid state imaging chip with a lens system disposed thereon.

19. The method of claim 18, wherein the image capture mechanism further comprises a rotational device coupled to a portion of the solid state imaging chip.

20. The method of claim 18, wherein the image plane of the solid state imaging chip is substantially perpendicular to a longitudinal axis of the imaging device.

21. An imaging device, comprising:
an elongated member, the elongated member having a distal end and a proximal end;
at least one solid state imaging chip disposed at the distal end of the elongated member, the at least one solid state imaging chip comprising at least one imaging array; and
an annular prism optically coupled to the at least one imaging array.

22. The imaging device of claim 21, wherein the annular prism is disposed in direct contact with the imaging array.

23. The imaging device of claim 22, further comprising a GRIN lens disposed in direct contact with the imaging array.

24. The imaging device of claim 23, wherein the annular prism comprises an aperture in the center of the annular prism.

25. The imaging device of claim 24, wherein the GRIN lens is disposed in the aperture of the annular prism.
